(19) United States
(12) Patent Application Publication
(10) Pub. No.: US 2004/0238371 A1
(43) Pub. Date: Dec. 2, 2004

(54) Coated method for light metal alloys

(76) Inventor: Wolf-Dieter Franz, Geretsried (DE)

Correspondence Address: COHEN, PONTANI, LIEBERMAN & PAVANE, 551 FIFTH AVENUE, SUITE 1210, NEW YORK, NY, 10176, US

(21) Appl. No.: 10/492,283

(22) PCT Filed: Oct. 9, 2002
(86) PCT No.: PCT/EP02/11292

(30) Foreign Application Priority Data
Oct. 11, 2001 (EP) 011244399

(51) Int. Cl. C25D 7/00; C23C 28/02; C07K 1/26

(52) U.S. Cl. 205/187; 204/484; 204/486

(57) ABSTRACT

A new coating method for light metal alloys in which an electrophoretic lacquering and a galvanic coating are combined. Thus, corrosion resistant surfaces having very good optical properties can be achieved, wherein an advantageous flexibility in view of material selection and layer thickness is preserved.

This invention relates to a coating method for light metal alloys. This term means alloys having Al, Mg or both metals as components being substantial for the surface properties.

The surface treatment of such light metal alloys implies special problems because these alloys are relatively reactive and especially sensitive to oxidation due to their contents of Al or Mg. On the other hand, light metal alloys are increasingly applied in a multitude of technical areas. This applies not only to classical applications such as air craft construction but also to novel applications such as vehicle components or casing components of high-grade devices. On the one hand, these light metal alloys are hard to be replaced in view of their low specific weight with concurrently good mechanical properties, as long as the complete mass plays a critical role in the application considered. A present example are casings of mobile phones. On the other hand, these technical applications are limited or at least hindered by the problems with the conservation and long lasting optical finishing of such light metal alloy parts.

In part, technically very demanding and thus expensive methods are used such as e. g. sputter coating methods. Further, such methods are frequently accompanied by additional limitations. E. g. sputter coating of large parts is extremely expensive or impossible. Further, only comparatively “open” part geometries can be used.

Further, many coating methods imply substantial problems in view of adhesion properties, corrosion resistance, and optical quality of the resulting surfaces.

Thus, the invention has the technical object to provide an advantageous coating method for light metal alloy surfaces.

The invention relates to a method for coating light metal alloy surfaces, comprising the steps:

electrophoretic lacquering of the light metal alloy surface and

galvanic coating of the electrophoretically lacquered surface.

Preferred embodiments of this method are defined in the dependent claims.

Precautionarily, it is further to be mentioned that the invention not only relates to the method steps but also to correspondingly coated parts. The applicant reserves the right to file claims of the product category. The following description is to be understood both in view of the method features and in view of the corresponding product features of the thus coated parts.

The idea underlying the invention is that the insufficient properties of galvanic layers on light metal alloys in view of optical quality and durability as well as corrosion protection can be improved dramatically by applying the galvanic layers on an electrophoretic lacquer layer. Surprisingly, it has been found that the electrophoretic lacquer layer does not principally hinder a later galvanic coating of the light metal alloy parts, and the advantages of these methods can thus be combined with each other.

Especially, the electrophoretic lacquering has the advantage to provide very tight and stable layers which thus provide a very good corrosion protection. Further, the electrophoretic lacquer layers have a good planarizing effect and thus provide a planarized substrate for the succeeding galvanic layer even in case of comparatively rough original surfaces of the light metal alloy. This improves the optical quality.

On the other hand, the galvanic coating has the advantage of an outstanding flexibility in view of layer arrangement, material selection, and layer thickness so that various technical requirements as roughness, abrasion resistance, hardness, conductivity and also aesthetic requirements can be taken into account.

Finally, the method according to the invention is also very flexible in view of size and geometry of the parts to be treated.

It has proven to be advantageous to activate the electrophoretically lacquered surface before the galvanic coating. Hereto, the classical activation steps of synthetic resin galvanics can be used. Preferably, a sulphonation (SO₃ atmosphere) or a treatment with chromic acid or another solution of hexavalent chrome can be used. Both treatments lead to a chemical alteration of the lacquered surface that improves the possibilities for an application of the galvanic layer.

Further, the layer properties are improved if the electrophoretic lacquer layer is chemically metallized e. g. by a chemical Ni layer or a chemical Cu layer. In case of use of both, the surface activation and the chemical metallization, the metallization is conducted after activation.

Further, the chemical metallization can be preceded by a metal seeding, especially a Pd activation, e. g. a treatment in a PdCl solution. Also this step succeeds an optional activation of the lacquered surface. A reduction step can be used between the Pd activation and the chemical metallization in order to provide metallic Pd.

The electrophoretic lacquering characteristic for the invention can be a cathodic or anodic immersion lacquering, wherein the cathodic immersion lacquering is especially preferred in case of high requirements in view of corrosion resistance. Further, the step of electrophoretic lacquering is conventional as such and is conducted by means of common materials and methods.

It is, however, preferred with the invention to provide an adhesion or bonding agent layer between the electrophoretic lacquer layer and the light metal alloy surface, i. e. to precede the electrophoretic lacquering by a corresponding coating step. As bonding agent layers, especially chromate layers, treatments with Zr fluoride solutions or ZrTi fluoride solutions or other conversion layers are considered. Especially preferred are oxidic layers, however, wherein the oxidic layers can comprise also phosphates or consist of phosphates. Especially it is considered to provide the electrophoretic lacquering on an anodic layer on the light metal alloy surface which comprises oxides and/or phosphates of the alloy components, thus namely of Al and/or Mg.

Especially good results can be achieved if the voltage used during the electrophoretic lacquering is chosen higher than the voltage used for the anodic bonding agent layer. The voltage can be higher than the voltage for the anodic bonding agent coating by 10% or more.

The invention has a preferred application on light metal alloy die-casting parts on which the electrophoretic lacquering produces a high degree of planarization and smoothness, if required, so that with a succeeding galvanic coating a high degree of gloss can be achieved. If this smooth surface is e. g. chromium plated, a very high-grade optical appearance results.

Preferred application fields are in casing parts of mobile phones and other mobile electronic apparatus as laptops, PDAs and the like, or of cameras, binoculars and other optical apparatus. Further, light metal alloys have increasing applications in vehicle technology. In many cases, an optical appearance resembling a classical chromium plating or other high-degree metal surfaces is favoured. Examples are door handles and other armature parts as well as rims.

The invention has special advantages on light metal alloys with a high Mg ratio since these show especially severe corrosion problems. On the other hand, these alloys are very interesting for technical reasons because of their exceptionally low specific weight. A preferred application is on light metal alloys having a Mg ratio of at least 50 weight %.

An embodiment of the invention is a casing part of a mobile phone of a Mg alloy being a chassis between two synthetic resin shells and being the basis for the mechanical stability of the mobile phone casing. The embodiment is a die-casting part showing as such at the beginning a rough surface and inadequate optical properties.

This casing part is first coated with an anodic oxidation/phosphatizing layer of 3-5 μm thickness being offered under the trade name “Anomag” by Magnesium Technology Licensing Ltd. (Auckland, New Zealand) and their contract partners. However, also a conventional Zr fluoride treatment or chromalizing is possible (e. g. of 0.5-2 μm). Thereafter, the mobile phone part is coated with a common cathodic immersion lacquering with about 10 μm layer thickness in a reactor which is activated in an SO₃ atmosphere. Thereafter, the surface of the die-casting part is planarized. The lacquer used is Freiotherm KTL spezial.

After a Pd seeding, a reduction step in amino borane, and an application of a conventional chemical Ni layer of 0.5-1 μm thickness, a galvanic Ni plating (10 μm), and thereafter Cr plating (1 μm) are applied producing a surface quality and durability that is directly comparable to chromium plated steel parts.

On the other hand, the mobile phone casing part has an exceptionally low weight and good mechanical rigidness due to using the Mg alloy. Tests with such parts have shown corrosion resistances with results over 500 hours salt spray test.

1. A method for coating light metal alloy surfaces comprising the steps:
electrophoretically lacquering said light metal alloy surface,
chemically metallizing the electrophoretically lacquered light metal alloy surface, and
galvanically coating the electrophoretically lacquered surface after said chemically metallizing step.

2. A method according to claim 1, in which said electrophoretically lacquered surface is activated before said chemically metallizing step and said galvanically coating step.

3. A method according to claim 2, in which said activation comprises a sulphonation step.

4. A method according to claim 2, in which said activation comprises a treatment with hexavalent chromium.

5. (Cancelled)

6. A method according to claim 1, in which said surface is metal-seeded before said chemical metallization, and if applicable before said activation.

7. A method according to claim 1, in which said electrophoretic lacquering is a cathodic immersion lacquering.

8. (Cancelled)

9. (Cancelled)

10. (Cancelled)

11. (Cancelled)

12. A method according to claim 1, in which said light metal alloy surface is a surface of a light metal die-casting part.

13. A method according to claim 1, in which an alloy having a Mg ratio of more than 50 weight % is coated.

14. A method according to claim 1, in which said light metal alloy surface is the surface of a vehicle component.

15. A method according to claim 14, in which said vehicle component is a rim.
